(19) United States
(12) Patent Application Publication
(10) Pub. No.: US 2011/0106162 A1
(43) Pub. Date: May 5, 2011

(54) Composite Connecting Elements for Spinal Stabilization Systems

(75) Inventors: Rodney R. Ballard, Lakeland, TN (US); Bryan S. Wilcox, Collierville, TN (US); Christopher M. Patterson, Olive Branch, MS (US); Julien J. Prevost, Memphis, TN (US); Christopher F. Scifert, Bartlett, TN (US); Robert M. Loke, Memphis, TN (US)

(73) Assignee: Warsaw Orthopedic, Inc., Warsaw, IN (US)

(21) Appl. No.: 12/609,871

(22) Filed: Oct. 30, 2009

(51) Int. Cl. A61B 17/70; A61B 17/88; B29C 63/22

(52) U.S. Cl. 606/254; 606/279; 264/279

(57) ABSTRACT

Elongated connecting elements include bodies having composite cross-sections defined by a center core that is surrounded by an outer portion. The center core includes a first material and the outer portion includes a second material that is distinct from the first material. In one particular form, a connecting element includes a maximum dimension across an outer cross-sectional shape of the outer portion that is less than a minimum dimension across an oblong or round outer cross-sectional shape of a polyetheretherketone (PEEK) connecting element, and the connecting element exhibits mechanical properties that are at least equivalent to the mechanical properties of the polyetheretherketone (PEEK) connecting element. In another form, the center core has a non-circular cross-sectional shape and the first material is defined by a reinforcing material randomly dispersed throughout a polymer, and the outer portion includes a circular, outer cross-sectional shape. However, other embodiments, forms and applications are also envisioned.

### BACKGROUND

Various devices and methods for stabilizing bone structures have been used for many years. For example, one type of stabilization technique uses one or more elongated rods extending between components of a bony structure and secured to the bony structure to stabilize the components relative to one another. The components of the bony structure are exposed and one or more bone engaging fasteners are placed into each component. The elongated rod is then secured to the bone engaging fasteners in order to stabilize the components of the bony structure.

In some instances, different elongated rods each having desired mechanical and/or material properties are provided, but are not readily engageable to a variety of bone engaging fasteners due to their respective outer dimensions or their external configurations.

### SUMMARY

Elongated connecting elements include bodies having composite cross-sections defined by a center core that is surrounded by an outer portion. The center core includes a first material and the outer portion includes a second material that is distinct from the first material. In one particular form, a connecting element includes a maximum dimension across an outer cross-sectional shape of the outer portion that is less than a minimum dimension across an oblong or round outer cross-sectional shape of a polyetheretherketone (PEEK) connecting element, and the connecting element exhibits mechanical and/or material properties that are at least equivalent to the mechanical and/or material properties of the polyetheretherketone (PEEK) connecting element.

According to one aspect, a connecting element for a spinal stabilization system includes an elongate body extending along a longitudinal axis between opposite first and second ends. The elongate body includes a composite cross-section with a center core that includes a non-circular cross-sectional shape and is comprised of a first material defined by a reinforcing material randomly dispersed throughout a polymer. An outer portion that includes a circular, outer cross-sectional shape is positioned around the center core and is comprised of a second material.

According to another aspect, a method includes producing an elongate connecting element for a spinal stabilization system. The connecting element includes an elongate body extending along a longitudinal axis and including a composite cross-section. The connecting element further exhibits mechanical properties that are at least equivalent to mechanical properties of a polyetheretherketone (PEEK) connecting element having an oblong or round outer cross-sectional shape. The method further includes providing a center core comprised of a first material and including a first cross-sectional shape, and positioning an outer portion around the center core. The outer portion includes a second material and has a maximum dimension across an outer cross-sectional shape that is less than a minimum dimension across the oblong outer cross-sectional shape of the polyetheretherketone (PEEK) connecting element.

According to another aspect, a method for spinal stabilization includes engaging an anchor to a first vertebral body. The anchor includes a receiver positioned adjacent the first vertebral body. The method also includes positioning a connecting element in the receiver of the anchor. The connecting element is produced by providing a center core comprised of a first material and including a first cross-sectional shape, and positioning an outer portion comprised of a second material around the center core. The connecting element includes an elongate body extending along a longitudinal axis and including a composite cross-section. The connecting element further exhibits mechanical properties that are at least equivalent to mechanical properties of a polyetheretherketone (PEEK) connecting element having an oblong or round outer cross-sectional shape, and the outer portion includes a maximum dimension across an outer cross-sectional shape that is less than a minimum dimension across the outer cross-sectional shape of the polyetheretherketone (PEEK) connecting element.

Related features, aspects, embodiments, objects and advantages of the present invention will be apparent from the following description.

### BRIEF DESCRIPTION OF THE DRAWINGS

FIG. 1 is a posterior view of a spinal fixation system relative to the spinal column of a patient.

FIGS. 2A and 2B are a perspective view and a section view, respectively, of an elongate connecting element suitable for use in the spinal fixation system illustrated in FIG. 1.

FIG. 3 is a section view of an alternative embodiment connecting element suitable for use in the spinal fixation system illustrated in FIG. 1.

FIG. 4 is an end view of a polyetheretherketone (PEEK) connecting element having an oblong outer cross-sectional shape.

FIGS. 5-11 show end views of various embodiments of composite connecting elements.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

A connecting element for connection with anchors engaged to one or more vertebral bodies is provided with a composite cross-section that extends along all or a substantial portion of a length of a body of the connecting element. In one embodiment, the composite cross-section is constant in dimension and material properties along the entire length of the connecting element. Other embodiments contemplate cross-sections that vary in dimension and/or material properties along all or a portion of the entire length of the connecting element.

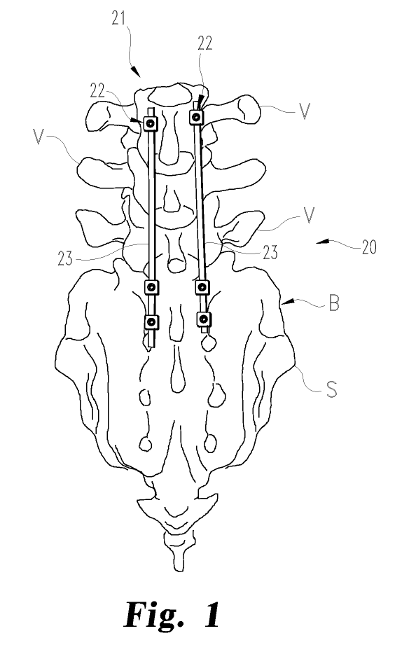

FIG. 1 illustrates a spinal fixation system 20 located at a desired skeletal location of a patient. More specifically, as depicted in FIG. 1, system 20 is affixed to bones B of the spinal column 21 from a posterior approach, although alternative approaches and/or locations of fixation of system 20, such as anterior, posterior mid-line, lateral, postero-lateral, and/or antero-lateral approaches, are contemplated. Bones B include the sacrum S and several vertebrae V. System 20 generally includes several bone anchors 22 and connecting elements 23 structured to selectively interconnect with bone anchors 22. In system 20, bone anchors 22 are affixed to various locations of the spinal column 21 and interconnected with connecting elements 23. Posterior fixation system 20 may be used for, but is not limited to, treatment of degenerative spondylolisthesis, fracture, dislocation, scoliosis, kyphosis, spinal tumor, and/or a failed previous fusion.

Each of anchors 22 includes a receiver for receiving connecting element 23 therein, and a bone engaging portion for engaging vertebrae V. The bone engaging portion can be a threaded screw-like member that extends into and engages the bony structure of vertebrae V. Other embodiments contemplate that one or more of anchors 22 can include a bone engaging portion in the form of a hook, staple, bolt, clamp, cable, or other suitable bone engaging device. In one form, the receiver can include a pair of arms defining a passage therebetween for receiving the connecting element 23 therebetween. The arms can be top-loading and internally and/or externally threaded to engage a set screw or other member to couple connecting element 23 with anchors 22. Other embodiments contemplate receivers that are side-loading, bottom loading, end-loading, clamping members, or any other suitable arrangement for securing connecting element 23 along the spinal column. The receiver can pivot or rotate relative to the bone engaging portion, or can be fixed relative to the bone engaging portion. In one embodiment, anchor 22 is a bone screw with a U-shaped head pivotally mounted or fixed to the proximal end of a bone screw.

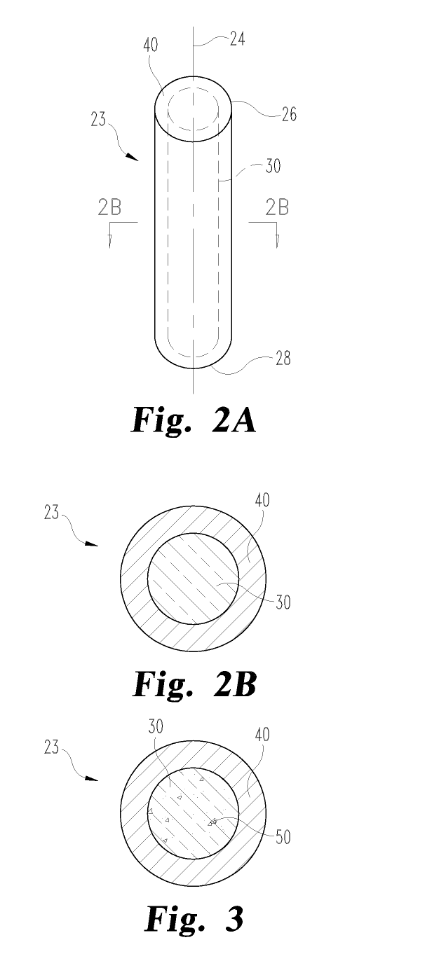

As further shown in FIG. 2A, connecting element 23 is elongated and extends along a central longitudinal axis 24 between opposite first and second ends 26, 28. Connecting element 23 includes a length from first end 26 to second end 28 that is sized to extend along one or more spinal motion segments that include two or more vertebrae and to allow connecting element 23 to be engaged to at least two anchors 22 engaged to respective ones of the at least two vertebrae. In the illustrated form, connecting element 23 has a substantially linear configuration, although it should be appreciated that it may also be curved or bent in one or more regions along longitudinal axis 24 to match or provide a contour of the spinal column 21. Connecting element 23 includes a composite cross-section with a core 30 and an outer portion 40 extending around core 30. As illustrated in FIG. 2B, core 30 and outer portion 40 each include a circular cross-sectional shape. It should be appreciated however that one or both of core 30 and outer portion 40 may be alternatively shaped, such as oval or oblong shaped, as will be discussed below in greater detail. In addition, in other forms, it is contemplated that connecting element 23 could be provided with two or more cores 30 positioned in outer portion 40, or that core 30 could be defined by two or more elements made from the same or different materials. In one form, core 30 is defined by a plurality of strands or fibers that are braided or otherwise bundled together. In another form, core 30 may be defined by two or more braided or bundled groups of fibers or strands, or may be defined by one or more braided or bundled groups of fibers or strands in combination with one or more unbraided or unbundled strands or fibers.

In one embodiment, the composite connecting element 23 is provided with core 30 made from a material having a higher modulus of elasticity and outer portion 40 made from a material having a lower modulus of elasticity than the material of core 30. In the form illustrated in FIG. 2B, core 30 is formed of a single material, although forms where core 30 is formed of a composite material are also contemplated as will be discussed in further detail below with respect to FIG. 3. Non-limiting examples of materials that could be used for all or part of core 30 include Grade 5 titanium (Ti-6Al-4V), Commercially Pure Titanium (CP Ti), cobalt-chromium (Co—Cr), stainless steel, Nitinol, carbon-fiber reinforced polyetheretherketone (PEEK) and/or glass-fiber reinforced polyetheretherketone (PEEK). In one form, examples of suitable material for outer portion 40 include those materials with a lower modulus of elasticity than that of the selected material of core 30, such as PEEK, polyurethane, epoxy, CP Ti, Nitinol and/or polyurethane-silicon copolymers such as Elast-Eon™. An outer portion 40 having a greater relative stiffness and a core 30 being more compliant are also contemplated in other embodiments.

Further examples of materials that may be used for all or part of core 30 include non-resorbable materials, cobalt-chrome alloys, titanium alloys, superelastic metallic alloys (for example, NITINOL®, GUM METAL®), stainless steel alloys, and/or reinforced members of the polyaryletherketone family, such as continuous carbon fiber reinforced PEEK, short carbon fiber reinforced PEEK, or shape-memory PEEK for example. Further examples of suitable materials for outer portion 40 include non-resorbable materials and/or members of the polyaryletherketone family. More particular examples of suitable materials for outer portion 40 include short carbon fiber reinforced PEEK, continuous carbon fiber reinforced PEEK, shape-memory PEEK, superelastic metal alloys, polyetherketoneketone (PEKK), polyethylene, polyphenylene, polysulfone, polyetherimide, polyimide, and/or ultra-high molecular weight polyethylene (UHMWPE).

In the embodiment of FIG. 3, where like numerals refer to like features previously described, core 30 is made from a composite material that includes a reinforcing material 50 which in the illustrated form is defined by a plurality of particles dispersed throughout, for example, a polymer. More particularly, in FIG. 3, the particles of reinforcing material 50 are randomly dispersed throughout core 30, and may be, for example, in the form of chopped carbon, metal fibers or glass fibers. However, in other forms it is contemplated that the particles of reinforcing material 50 may be aligned or oriented in a common direction to provide core 30 with desired mechanical or material properties. In another form, reinforcing material 50 may be provided as one or more continuous fibers that extend along the length of connecting element 23 between first and second ends 26, 28. Still, in other forms it is contemplated that reinforcing material 50 could include a plurality of braided or otherwise arranged fibers to provide core 30 with desired mechanical or material properties. In addition, while not previously described, it should be appreciated that only certain portions of core 30 between first and second ends 26, 28 may be provided with reinforcing material 50 such that the mechanical and/or material properties of connecting element 23 vary between ends 26, 28. Reinforcing material 50 may be formed of one or more metals or metallic materials, a polymer, such as PEKK or shape-memory PEEK, carbon fiber, or glass fiber, just to provide a few non-limiting examples. In addition, in other non-illustrated forms, connecting element 23 can be provided with core 30 and outer portion 40 that are each comprised of a composite material. For example, in one specific embodiment, core 30 is made of composite material such as short carbon fiber in PEEK, and the outer portion 40 is made of a composite material such as short carbon fiber in PEEK that has a lower percentage of short carbon fiber than the material of core 30.

Another embodiment composite connecting element includes a circular, oval, elliptical, oblong, racetrack, or rectangular core made from Ti-6Al-4V and a circular outer layer around the core made from PEEK. Another example composite connecting element includes a circular, oval, elliptical, oblong, racetrack or rectangular core made from Ti-6Al-4V and a circular outer layer around the core made from polyurethane. Another embodiment composite connecting element includes a circular, oval, elliptical, oblong, racetrack, or rectangular core made from Co—Cr and a circular outer layer around the core made from PEEK. Another example composite connecting element includes a circular, oval, elliptical, oblong, racetrack or rectangular core made from Nitinoland a circular outer layer around the core made from silicone. Another embodiment composite connecting element includes a circular, oval, elliptical, oblong, racetrack, or rectangular core made from stainless steel and a circular outer layer around the core made from epoxy. Another embodiment composite connecting element includes a circular, oval, elliptical, oblong, racetrack, or rectangular core made from carbon-fiber or glass-fiber reinforced PEEK and a circular outer layer around the core made from PEEK. Still, other variations in the composition and shape of core 30 and outer portion 40 are contemplated.

As illustrated in FIGS. 2B and 3, outer portion 40 radially surrounds core 30 and prevents exposure of core 30 to the environment surrounding connecting element 23. In FIG. 2A, core 30 is generally illustrated as being exposed at ends 26, 28 of connecting element 23, although it should be appreciated that in alternative forms outer portion 40 may also surround and cover core 30 at ends 26, 28. Among other things, outer portion 40 provides a corrosion barrier between core 30 and other elements, such as anchors 22, of system 20, contains any debris that may wear off of core 30, and prevents notches and other imperfections in core 30 that may result from the placement and arrangement of connecting element 23 relative to anchors 22. In one form, outer portion 40 may be provided as a sleeve that is heat shrunk over core 30, or may be injected with the material of core 30. In other forms, the arrangement of core 30 and outer portion 40 of connecting element 23 may be provided by pultrusion, co-extrusion, overmolding, compression molding and injection molding, just to provide a few possibilities.

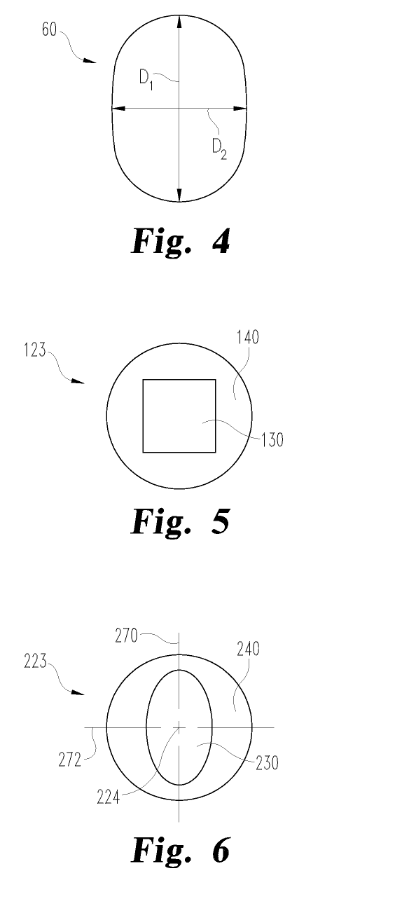

FIG. 4 shows an end view of a solid, polyetheretherketone (PEEK) connecting element 60 having an oblong outer cross-sectional shape. Connecting element 60 generally includes, across its oblong outer cross-sectional shape, a maximum dimension D₁ between rounded end segments and a minimum dimension D₂ between elongate sides extending between the end segments. In one particular form, maximum dimension D₁ is 7.14 millimeters and minimum dimension D₂ is 6.38 millimeters. In other forms, connecting element 60 may have a circular cross-sectional shape such that its diameter defines both the maximum dimension D₁ and minimum dimension D₂. It has now been surprisingly discovered that the composite connecting elements disclosed in this document can be provided with a maximum outer dimension taken across the external surfaces thereof that is less that than the minimum dimension D₂ of connecting element 60, whether oblong or round, while still exhibiting mechanical and/or material properties in all directions that are at least equivalent to those of connecting element 60, although in some embodiments the composite connecting elements disclosed in this document can exhibit mechanical and/or material properties superior to those of connecting element 60. With particular regard to the forms illustrated in FIGS. 2B and 3 for example, the diameter of connecting element 23 having a circular cross-section shape is less than 6.38 millimeters. In one more particular form, the diameter of connecting element 23 having a circular cross-sectional shape is 4.75 millimeters, although other values for the diameter of connecting element 23 are contemplated. Moreover, in other forms where connecting element 23 includes a non-circular cross-sectional shape, it should be appreciated that the maximum outer dimension thereof taken across its external surfaces will also be less than the minimum dimension D₂ of connecting element 60.

FIGS. 5-11 show various shapes for the cross-sections or ends of various embodiments of the connecting elements discussed herein. In each of FIGS. 5-10, outer portion 40 includes a circular outer shape and provides an isotropic cross-section and therefore is not constrained by the anchor 22 with respect to the rotational positioning of the connecting element 23 about longitudinal axis 24. In addition, in each of FIGS. 6-10, the connecting element 23 includes a non-circular core that provides connecting element 23 with two or more bending axes which, in at least some embodiments, include different bending stiffnesses thereabout. In this configuration, connecting element 23 can be rotated relative to spinal column 21 to provide a desired resistance to certain spinal segment motion, such as extension or flexion for example. Further details regarding these configurations and their use relative to the spinal column are provided in the commonly-owned U.S. patent application entitled “Selectable Flexion Rods” and assigned docket number MSDI-1166/P35171, the contents of which are incorporated herein by reference in their entirety.

Referring now to FIG. 5, connecting element 123 includes a core 130 with a square cross-sectional shape that is surrounded by outer portion 140. In FIG. 6, connecting element 223 includes a core 230 with an oval cross-sectional shape that has a major dimension extending along first bending axis 270 and a minor dimension extending along second bending axis 272. Bending axes 270, 272 extend 90 degrees relative to another, allowing the bending resistance of connecting element 223 to be varied between its most stiff and least stiff orientations through a quarter turn of connecting element 223 about longitudinal axis 224.

FIG. 7 shows another embodiment composite connecting element 323 that includes a circular outer portion 340 and a core 330 with an oblong or race-track cross-sectional shape. Core 330 includes rounded ends at its major dimension defining a first bending axis 370, and linear sides extending between the rounded ends that define a minor dimension extending along second bending axis 372. Bending axes 370, 372 extend 90 degrees relative to another, allowing the bending resistance of connecting element 323 to be varied from its most stiff to its least stiff orientation through a quarter turn of connecting element 323 about longitudinal axis 324 to align axis 370 or axis 372 in the direction of bending. Intermediate axes between bending axes 370, 372 can also be aligned in the direction of bending to provide an intermediate bending stiffness.

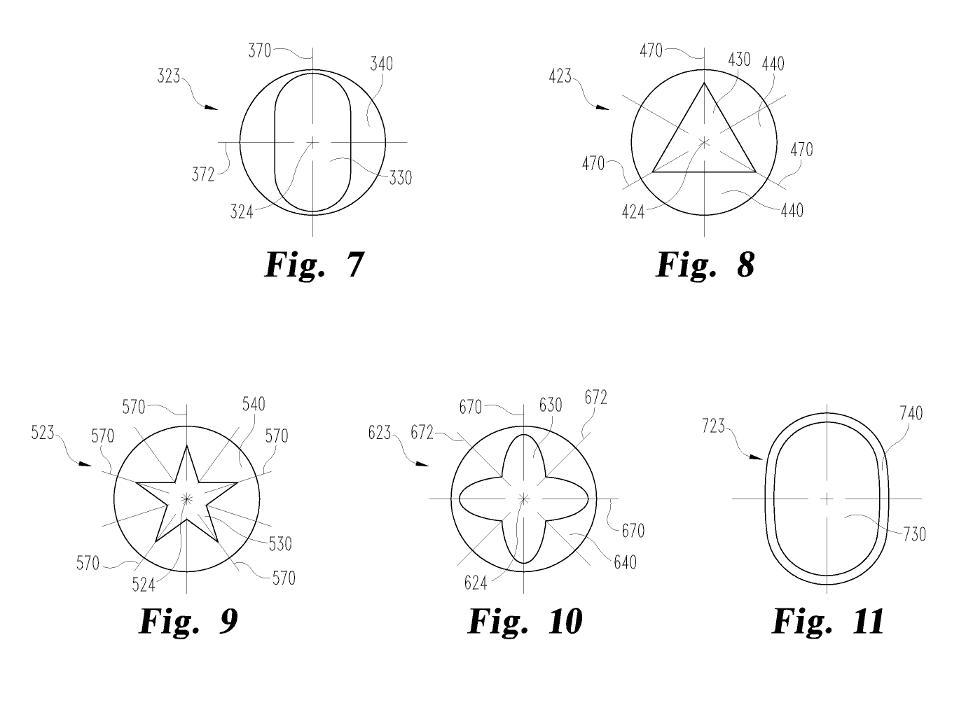

FIG. 8 shows another embodiment composite connecting element 423 that includes a circular outer portion 440 and a core 430 with a triangular cross-sectional shape. Core 430 includes a major dimension extending through each vertex to the middle of the opposite side of the triangular shape, defining three major bending axes 470. Connecting element 423 provides greatest resistance to bending forces when one of the major bending axes 470 is aligned with the direction of bending. Resistance to bending forces is reduced by rotating connecting element 423 about longitudinal axis 424 to a location aligning an intermediate axis located between major bending axes 470 in the direction of bending.

FIG. 9 shows another embodiment composite connecting element 523 that includes a circular outer portion 540 and a core 530 with a star-shaped cross-section. Core 530 includes a major dimension extending through each vertex of the star, defining five major bending axes 570. Connecting element 523 provides greatest resistance to bending forces when one of the major bending axes 570 is aligned with the direction of bending. Resistance to bending forces is reduced by rotating connecting element 523 about longitudinal axis 524 to a location between major bending axes 570.

FIG. 10 shows another embodiment composite connecting element 410 that includes a circular outer portion 640 and a core 630 with a shape formed by four rounded, interconnected lobes. Core 630 includes rounded ends at the major dimension of opposite ones of the lobes defining two major bending axes 670. The location intermediate the adjacent lobes provides a minor dimension of the core and defines minor bending axes 672. Bending axes 672, 670 extend about 45 degrees relative to another, allowing the bending resistance of connecting element 623 to be varied between its most stiff and least stiff orientations through an eighth turn of connecting element 623 about longitudinal axis 624.

As suggested above, it is contemplated that the composite connecting elements disclosed herein could be provided with outer portions that include non-circular cross-sectional shapes. More particularly, in one or more forms, it is contemplated that the outer portion could have a cross-sectional shape that corresponds to the cross-sectional shape of the core, including for example any of the shapes illustrated in FIGS. 5-10 or otherwise discussed herein. For example, FIG. 11 shows another embodiment composite connecting element 723 that includes a race-track cross-sectional shape outer portion 740 and a core 730 with a corresponding race-track cross-sectional shape.

In one aspect, the connecting elements described herein include composite cross-sections that allow a surgeon to use a connecting element having a maximum external dimension that that is smaller than the minimum external dimension of an oblong or round shape connecting element formed only of polyetheretherketone (PEEK). In another aspect, the connecting elements disclosed herein allow the surgeon intra-operative freedom to select or adjust the flexion-extension stiffness of the connecting element by selecting the bending axis that is aligned in the direction of bending of the spinal motion segment. In one form, the connecting elements include an outer round or circular profile having a configuration suitable for engagement with a variety of different bone anchors. In one or more forms, the composite connecting elements include a non-circular core that is centrally located in the outer portion, although offset locations of the core relative to the outer portion are contemplated. When utilized, the non-circular cross-section of the core allows the stiffness of the rod in a particular plane of patient motion to be selected or adjusted during implantation or manufacture by changing the orientation of the core relative to the selected plane. In one specific example, the plane of motion of the patient is flexion and extension motion in the sagittal plane of a spinal motion segment including two or more vertebrae.

In one embodiment, the core of the connecting elements is made from a higher modulus material and the outer portion is made from a lower modulus material. The connecting element may be linear and straight along its entire length, or may be curved along all or part of its length. The connecting element may be pre-shaped, or shaped in the operating room or in situ. The connecting elements can be manufactured with various manufacturing processes, including over-molding the outer portion on the core, injection molding, extrusion, compression molding, or casting, for example. The core may also include surface treatments, such as shot-peening, grit-blasting, texturing, plasma treatment, anodizing or adhesive, for example, to facilitate and maintain engagement between the outer portion and the core. The composite structures discussed herein also have application with other types of implants, such as screws, plates, or cages, and may be used in other portions of the body. The connecting elements described herein may also be used in surgical procedures involving animals, or in demonstrations for training, education, marketing, sales and/or advertising purposes. In addition, the connecting elements may be also used on or in connection with a non-living subject such as a cadaver, training aid or model, or in connection with testing of surgical systems, surgical procedures, orthopedic devices and/or apparatus.

In certain embodiments, the area of the cross-section and/or the shape of the cross-section of the core of the composite connecting element is constant along the entire length of the connecting element. In other embodiments, the area of the cross-section and/or the shape of the cross-section of the core of the composite connecting element varies along the length of the connecting element. The outer portion surrounding the core may be solid, continuous, non-continuous, braided, knitted, or woven, for example. In addition, the outer portion may be of a composite material or contain any suitable additive.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A connecting element for a spinal stabilization system, comprising an elongate body extending between opposite first and second ends, said elongate body including a composite cross-section with a center core that includes a non-circular cross-sectional shape and is comprised of a first material defined by a reinforcing material randomly dispersed throughout a polymer, and an outer portion that includes a circular, outer cross-sectional shape and is positioned around said center core and comprised of a second material.

2. The connecting element of claim 1, wherein said first material has a first modulus of elasticity and said second material has a second modulus of elasticity that is less than said first modulus of elasticity.

3. The connecting element of claim 1, wherein said reinforcing material comprises carbon fiber and said polymer comprises polyetheretherketone (PEEK).

4. The connecting element of claim 3, wherein said second material is selected from the group consisting of a polyaryletherketone (PAEK), polysulfone, polyetherimide, polyimide, ultra-high molecular weight polyethylene (UHMWPE), and a polyurethane-silicone copolymer.

5. The connecting element of claim 4, wherein said polyaryletherketone (PAEK) is selected from the group consisting of polyetheretherketone (PEEK) and polyetherketoneketone (PEKK).

6. The connecting element of claim 1, wherein said reinforcing material comprises fiberglass and said polymer comprises polyetheretherketone (PEEK).

7. The connecting element of claim 1, wherein said non-circular cross-sectional shape of said center core is selected from the group consisting of oval, racetrack, triangular, star and multi-lobed shapes.

8. A method, comprising:
producing an elongate connecting element for a spinal stabilization system, said connecting element including an elongate body extending along a longitudinal axis and including a composite cross-section, said connecting element further exhibiting mechanical properties that are at least equivalent to mechanical properties of a polyetheretherketone (PEEK) connecting element having an oblong or round outer cross-sectional shape, wherein said producing includes:
providing a center core comprised of a first material and including a first cross-sectional shape; and
positioning an outer portion around said center core, said outer portion comprising a second material and including a maximum dimension across an outer cross-sectional shape that is less than a minimum dimension across said outer cross-sectional shape of said polyetheretherketone (PEEK) connecting element.

9. The method of claim 8, wherein said outer cross-sectional shape of said outer portion is circular.

10. The method of claim 8, wherein said first cross-sectional shape of said inner core corresponds to said outer cross-sectional shape of said outer portion.

11. The method of claim 10, wherein said first cross-sectional shape of said inner core and said outer cross-sectional shape of said outer portion are circular.

12. The method of claim 8, wherein said first material is selected from the group consisting of titanium, stainless steel, cobalt-chrome, carbon fiber reinforced PEEK, and glass-fiber reinforced PEEK.

13. The method of claim 12, wherein said second material is selected from the group consisting of a polyaryletherketone (PAEK), polysulfone, polyetherimide, polyimide, ultra-high molecular weight polyethylene (UHMWPE), and a polyurethane-silicone copolymer.

14. The method of claim 8, wherein said first cross-sectional shape of said inner core is non-circular and said outer cross-sectional shape of said outer portion is circular.

15. The method of claim 14, wherein said first cross-sectional shape of said inner core is selected from the group consisting of oval, racetrack, triangular, star and multi-lobed shapes.

16. The method of claim 14, wherein said first material is defined by a reinforcing material randomly dispersed throughout a polymer.

17. The method of claim 16, wherein said polymer comprises polyetheretherketone (PEEK) and said reinforcing material is selected from the group consisting of carbon fiber, glass fiber, metal fibers, braided metal, PEKK and shape-memory PEEK.

18. The method of claim 8, wherein said minimum dimension across said outer cross-sectional shape of said polyetheretherketone (PEEK) connecting element is 6.35 millimeters.

19. A method for spinal stabilization, comprising:
engaging an anchor to a first vertebral body, wherein said anchor includes a receiver positioned adjacent the first vertebral body; and
positioning a connecting element produced in accordance with the method of claim 7 in said receiver of said anchor.

20. The method of claim 19, which further includes locking said connecting element in said receiver.
